(19) United States
(12) Patent Application Publication
(10) Pub. No.: US 2026/0263589 A1
(43) Pub. Date: Sep. 10, 2026

(54) PORCINE EPIDEMIC DIARRHEA VIRUS VACCINE

(71) Applicant: UIF (University Industry Foundation), Yonsei University, Seoul (KR)

(72) Inventor: Jae-Chul PYUN, Seoul (KR)

(73) Assignee: UIF (University Industry Foundation), Yonsei University, Seoul (KR)

(21) Appl. No.: 19/550,913

(22) Filed: Feb. 26, 2026

(30) Foreign Application Priority Data
Feb. 26, 2025 (KR) 10-2025-0025119

(51) Int. Cl. A61K 39/215; A61K 31/14; A61K 39/00; A61K 39/385; C07K 14/005; C12N 7/00

(52) U.S. Cl. CPC A61K 39/215; A61K 31/14; A61K 39/385; C07K 14/005; C12N 7/00; A61K 2039/552; A61K 2039/575; A61K 2039/6031; C12N 2770/20022; C12N 2770/20034

(57) ABSTRACT

A vaccine for porcine epidemic diarrhea virus is disclosed. The vaccine may comprise peptides having the sequences of an amino acid sequence selected from the group consisting of: (a) the amino acid sequence of SEQ ID NO: 1; (b) the amino acid sequence of SEQ ID NO: 2; (c) the amino acid sequence of SEQ ID NO: 3; and (d) the amino acid sequence of SEQ ID NO: 4.

### CROSS-REFERENCE TO RELATED APPLICATION

This application claims priority from Korean Patent Application No. 10-2025-0025119, filed on Feb. 26, 2025, in the Korean Intellectual Property Office, and all the benefits accruing therefrom under 35 U.S.C. 119. The contents of the above application are incorporated herein in its entirety by reference.

### INCORPORATION-BY-REFERENCE OF MATERIAL SUBMITTED BY U.S.P.T.O. PATENT CENTER

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_1710950034_SequenceListing,” is created on Feb. 26, 2026, and contains a 9,868 bytes file.

### BACKGROUND

### Field

The present invention relates to a vaccine for porcine epidemic diarrhea virus (PEDV).

### Description of Related Art

Porcine epidemic diarrhea virus (PEDV) is an RNA virus that primarily infects pigs and is a member of the porcine coronavirus family. The main route of infection is oral transmission, and the virus can spread through contaminated water, feed, equipment, and the like. Infection with PEDV causes diarrhea, vomiting, and dehydration, and results in a particularly high mortality rate in neonatal piglets. These symptoms directly impact farm productivity and can lead to significant economic losses.

Vaccines against PEDV can help reduce viral spread; however, continuous research and new approaches are required due to issues such as the emergence of variants and insufficient durability of immunity. Accordingly, there is a need for new vaccine strategies capable of inducing a more effective immune response.

An Fv antibody is a fragment constituting the variable region of an antibody and has the ability to bind to a specific site of an antigen. The Fv antibody consists of variable regions of a light chain and a heavy chain and is primarily responsible for binding to an antigenic epitope. Due to its small size, structural flexibility, and high affinity for specific antigens, Fv antibodies can be advantageously used in various biological applications. In particular, they can selectively bind antigens in viral-targeted therapeutics and diagnostic technologies, and can also be effectively applied as tools for antigen discovery and screening in vaccine development.

Auto-display technology is a technique for expressing specific proteins on the surface of bacterial cells, typically based on an Escherichia coli system. By using this technology, foreign proteins or antibody fragments can be exposed on the cell surface, enabling direct binding with antigens. This approach is useful in library screening and antibody discovery processes and facilitates efficient isolation and characterization of desired antibodies. In addition, auto-display systems have relatively low costs and are suitable for large-scale production, making them promising for application in vaccine development and discovery of new therapeutics.

### SUMMARY

A problem to be solved by the present invention is to overcome limitations in the development of vaccines capable of inducing an effective immune response against porcine epidemic diarrhea virus (PEDV). Conventional vaccines have limitations in responding to mutated viruses or maintaining long-term immunity. In addition, antibody production for infection prevention may occur in an imbalanced manner, resulting in inconsistent protective efficacy.

To address these problems, the present invention provides a strategy capable of increasing selective binding to specific antigenic sites, thereby improving the quality and durability of immune responses. This may contribute to establishing a more stable and efficient immune defense system for prevention of PEDV infection.

In one aspect, the present invention provides a peptide capable of inducing production of antibodies against porcine epidemic diarrhea virus (PEDV) in vivo.

In one embodiment, the peptide may comprise one or more peptide sequences selected from the group consisting of the peptide sequence of SEQ ID NO: 1, the peptide sequence of SEQ ID NO: 2, the peptide sequence of SEQ ID NO: 3, and the peptide sequence of SEQ ID NO: 4.

In another aspect, the present invention provides a nucleic acid encoding the peptide.

In another aspect, the present invention provides a recombinant expression vector comprising the nucleic acid.

In another aspect, the present invention provides a cell transformed with the recombinant expression vector.

In one embodiment, the cell may comprise one or more cells selected from the group consisting of an animal cell, a plant cell, yeast, Escherichia coli, and an insect cell.

In one embodiment, the cell may comprise one or more cells selected from the group consisting of COS7 (monkey kidney) cells, NSO cells, SP2/0 cells, Chinese hamster ovary (CHO) cells, WI38 cells, baby hamster kidney (BHK) cells, MDCK cells, myeloma cell lines, HuT78 cells, HEK293 cells, Escherichia coli, Bacillus subtilis, Streptomyces sp., Pseudomonas sp., Proteus mirabilis, Staphylococcus sp., Aspergillus sp., Pichia pastoris, Saccharomyces cerevisiae, Schizosaccharomyces sp., and Neurospora crassa.

In another aspect, the present invention provides a vaccine material for porcine epidemic diarrhea virus, comprising:
a ferritin protein; and
a peptide bound to an outer surface of the ferritin protein.

In one embodiment, the peptide may be the peptide according to the embodiments described above.

In one embodiment, the ferritin protein may comprise an H-ferritin protein.

In another aspect, the present invention provides a vaccine composition for porcine epidemic diarrhea virus comprising the vaccine material.

In another aspect, the present invention provides a method for immunizing against porcine epidemic diarrhea virus, comprising administering the vaccine composition to an animal.

The present invention provides the possibility of more efficiently inducing an immune response against porcine epidemic diarrhea virus (PEDV). In particular, by combining a ferritin complex platform with an Fv antibody-based antigen discovery strategy, higher levels of neutralizing antibody production may be promoted compared to conventional vaccines. Such immune activation may contribute not only to infection prevention but also to slowing disease progression. In addition, targeting diverse antigenic sites may provide the potential to respond to viral mutations.

### BRIEF DESCRIPTION OF DRAWINGS

FIG. 1A is a schematic diagram showing a construct in which a receptor-binding domain (RBD) antigen of PEDV corresponding to amino acids 617-745 of the porcine epidemic diarrhea virus spike protein (UniProt ID: Q91AV1, total length 1,384 aa) derived from CV777 and KPEDV-9 strains is selected and expressed by co-expression with enhanced green fluorescent protein (eGFP). The selected RBD is a 200-mer protein of approximately 21.2 kDa that corresponds to the region binding to the host receptor APN.

FIG. 1B illustrates the spatial arrangement of the NTD, RBD, and CTD of the S1 region and the HR1, fusion peptide, and HR2 of the S2 region in the monomeric structure of the PEDV spike protein, together with the overall structural organization of the spike trimer formed by such monomers.

FIG. 1C shows expression of an sfGFP-RBD fusion protein using an intracellular expression plasmid in which the PEDV spike protein-derived RBD is fused to sfGFP and a His×6 tag, and SDS-PAGE analysis confirming an expression product of approximately 49 kDa.

FIG. 1D shows sequence information highlighting the positions of the S1 and S2 regions within the full-length PEDV spike protein amino acid sequence, with emphasis on the RBD region corresponding to amino acids 617-745.

FIG. 2A illustrates a screening process using a GFP-labeled TBD probe, in which cells displaying diverse Fv antibody libraries on their surfaces are shown on the left, and antigen labeled with fluorescence is bound to selectively detect antibody clones on the right.

FIG. 2B shows results of the screening experiment using the GFP-labeled TBD probe, in which Fv antibody candidate clones binding to the RBD antigen are selected based on fluorescence intensity distribution and classified through sequence analysis.

FIG. 2C shows comparative analysis of oligonucleotide sequences and corresponding CDR3 amino acid sequences of anti-RBD Fv antibody clones selected through the screening experiment using the GFP-labeled TBD probe.

FIG. 3A illustrates a process of producing Fv antibodies by cloning screened Fv variant sequences, including CDR1, CDR2, and CDR3 regions, into an expression vector, followed by fusion expression with sfGFP and purification to produce Fv antibodies specifically binding to the PEDV spike protein.

FIG. 3B shows SDS-PAGE analysis confirming expression of Fv antibodies specific to the PEDV spike protein (anti-PEDV SP), namely α-RBD-1, α-RBD-2, α-RBD-3, and α-RBD-4.

FIG. 4A shows binding analysis between Fv-anti-RBD-1 and the RBD antigen measured as an SPR signal over time using an SPT biosensor, and calculation of a binding affinity (K_{D}) of approximately 142 nM from concentration-dependent binding curves.

FIG. 4B shows binding analysis between Fv-anti-RBD-2 and the RBD antigen, with a calculated K_{D} of approximately 149 nM.

FIG. 4C shows binding analysis between Fv-anti-RBD-3 and the RBD antigen, with a calculated K_{D} of approximately 101 nM.

FIG. 4D shows binding analysis between Fv-anti-RBD-4 and the RBD antigen, with a calculated K_{D} of approximately 83 nM.

FIG. 4E shows quantitative comparison of limit of detection (LOD) and binding affinity (K_{D}) of the selected Fv antibodies α-RBD-1, α-RBD-2, α-RBD-3, and α-RBD-4, compared with known binding affinity between PEDV RBD and the APN receptor and the lack of binding to ACE2.

FIG. 5A illustrates the location of the RBD region bound by Fv-anti-RBD-1 in the monomeric PEDV spike protein structure (UniProt ID: Q91AV1) and key amino acid residues involved in binding.

FIG. 5B illustrates the binding location and key residues for Fv-anti-RBD-2.

FIG. 5C illustrates the binding location and key residues for Fv-anti-RBD-3.

FIG. 5D illustrates the binding location and key residues for Fv-anti-RBD-4.

FIG. 6A shows identification of antigen-binding residues and structural positions of anti-RBD Fv1 and schematic design of immunogenic sequences including peptide spacing and linker sequences.

FIG. 6B shows immunogenic sequence and linker design based on binding residues and distance information for anti-RBD Fv2.

FIG. 6C shows immunogenic sequence and linker design for anti-RBD Fv3.

FIG. 6D shows immunogenic sequence and linker design for anti-RBD Fv4.

FIG. 7A illustrates a nanoparticle complex in which human ferritin heavy chain (hFTH, 192-mer, 24.8 kDa) serves as a core and antigens 1-4 (each approximately 2.4 kDa) are displayed on the surface via a (G2S) 10 linker, forming a nanoparticle complex of approximately 46 kDa.

FIG. 7B schematically illustrates an antigen module in which antigens are repeatedly linked by a (G2S) 10 linker and fused with hFTH, and a comparative configuration in which Fc is fused to hFTH.

FIG. 7C compares linker-containing RBD antigen sequence constructs designed based on amino acid sequences and distance information of RBD antigens 1~4 bound to ferritin H.

FIG. 8A shows a transmission electron microscopy (TEM) image of a Ferritin RBD1 nanoparticle and particle size distribution analysis indicating an average size of approximately 30.6 nm.

FIG. 8B shows a TEM image of a Ferritin RBD2 nanoparticle with an average size of approximately 35.9 nm.

FIG. 8C shows a TEM image of a Ferritin RBD3 nanoparticle with an average size of approximately 31.6 nm.

FIG. 8D shows a TEM image of a Ferritin RBD4 nanoparticle with an average size of approximately 33.9 nm.

FIG. 9A is a diagram comparing particle size distribution histograms and average particle diameters for each of Ferritin complexes RBD1 to RBD4.

FIG. 9B is a diagram comparing immunogenic sequences, net charge at pH 7.4, and zeta potential for each of Ferritin-RBD-1 to Ferritin-RBD-4.

FIG. 10A is a diagram comparing optical density values of antibody responses measured by ELISA at the harvest time point after mice were immunized three times with Ft-pRBD-1 to Ft-pRBD-4 and Ft-pRBD-W.

FIG. 10B is a diagram comparing antibody titers of IgG1, IgG2a, IgG2b, and IgG3 subclasses induced in mice immunized with Ft-pRBD-1 to Ft-pRBD-4 and Ft-pRBD-W.

FIG. 11A is a diagram comparing IL-4 secretion measured in mice immunized with Ft-pRBD-1 to Ft-pRBD-4 and Ft-pRBD-W with a control group.

FIG. 11B is a diagram comparing IL-12 secretion measured in mice immunized with Ft-pRBD-1 to Ft-pRBD-4 and Ft-pRBD-W with a control group.

FIG. 11C is a diagram comparing IFN-γ secretion measured in mice immunized with Ft-pRBD-1 to Ft-pRBD-4 and Ft-pRBD-W with a control group.

FIG. 11D is a diagram comparing TNF-α secretion measured in mice immunized with Ft-pRBD-1 to Ft-pRBD-4 and Ft-pRBD-W with a control group.

FIG. 12 is a diagram showing CD4+/CD8+ T-cell analysis after ferritin complex vaccination, comparing the proportions of CD4-positive and CD8-positive cells in control mice and mice immunized with Ft-pRBD-1 to Ft-pRBD-4 and Ft-pRBD-W using flow cytometry.

FIG. 13 is a diagram comparing wells in which cytopathic effects occurred in Vero cells due to PEDV infection with wells in which PEDV infection was inhibited and Vero cells survived.

FIG. 14 is a diagram illustrating calculation of the 50% endpoint using the Reed-Muench method by determining cumulative positive and cumulative negative rates based on positive and negative results for each well against diluted PEDV.

FIG. 15A is a diagram showing neutralization assay results in which sera from infected patients 1, 2, and 3 were serially diluted and protection of cells against viral infection was observed (PEDV: 200 TCID₅₀/0.1 ml).

FIG. 15B is a diagram showing results comparing cell protection effects due to virus neutralization at each serum dilution using Ferritin RBD1 to RBD4 immune sera.

FIG. 16A is a diagram showing neutralization efficacy curves comparing relative viral infection rates according to serum dilution when Ferritin RBD1 to RBD4 and full-length RBD were used as immunogens (PEDV: 200 TCID₅₀/0.1 ml).

FIG. 16B is a diagram showing serum neutralizing antibody titers on a logarithmic scale compared with PBS and ferritin-only control groups when Ferritin RBD1 to RBD4 and full-length RBD were used as immunogens.

FIG. 17A is a schematic diagram showing expression and purification of Fv variant proteins fused with sfGFP after cloning selected Fv antibody variants into an expression vector.

FIG. 17B is a diagram showing confirmation of expression of α-RBD-1 to α-RBD-4 Fv variant proteins by SDS-PAGE and Western blot analysis.

FIG. 18A is a diagram showing microscopic images comparing inhibition of virus-induced cytopathic effects in cell culture when anti-RBD1 to anti-RBD4 Fv were treated at various concentrations.

FIG. 18B is a graph showing virus neutralization efficacy of each antibody by comparing changes in relative infection rates when anti-RBD1 to anti-RBD4 Fv were treated at various concentrations (PEDV: 200 TCID₅₀/0.1 ml).

FIG. 18C is a diagram comparing minimum neutralizing concentrations required to neutralize viral infection for each of anti-RBD1 to anti-RBD4 Fv.

FIG. 19 is a diagram schematically illustrating construction of an expression vector and purification process for the heavy chain variable region (VH) of anti-RBD antibodies, and showing SDS-PAGE results confirming approximately 50 kDa purified anti-RBD1 to anti-RBD3 VH proteins.

FIG. 20A is a schematic diagram showing analysis of antigen-antibody interaction by binding antibody heavy chains to a Z-domain-immobilized gold chip surface followed by association and dissociation of the PEDV RBD antigen.

FIG. 20B is a diagram showing analysis of binding affinity (K_{D} 40 nM) based on SPR signal changes when Anti-RBD1 VH expressed from a pFUSE heavy chain vector binds to the PEDV RBD antigen.

FIG. 20C is a diagram showing analysis of binding affinity (K_{D} 183 nM) for Anti-RBD2 VH.

FIG. 20D is a diagram showing analysis of binding affinity (K_{D} 88 nM) for Anti-RBD3 VH.

FIG. 21A is a diagram showing microscopic images comparing inhibition of cytopathic effects in PEDV-infected cells when Anti-RBD1 VH, Anti-RBD2 VH, and Anti-RBD3 VH were treated at different concentrations (PEDV: 200 TCID₅₀/0.1 ml).

FIG. 21B is a diagram comparing inhibition of relative PEDV infection rates according to concentration changes of Anti-RBD1 VH, Anti-RBD2 VH, and Anti-RBD3 VH.

FIG. 21C is a diagram comparing minimum neutralizing concentrations required for PEDV neutralization by Anti-RBD1 VH, Anti-RBD2 VH, and Anti-RBD3 VH.

FIG. 22 is a diagram illustrating mammalian cell transformation, antibody expression, and IgG purification using a pTRIOZ plasmid comprising a heavy chain specific for PEDV RBD2 and a light chain specific for PEDV RBD1.

FIG. 23A is a diagram illustrating analysis of binding and dissociation between a PEDV RBD antigen fused with sfGFP and an antibody comprising the V_{H}-RBD2 and V_{L}-RBD1 combination expressed via pTRIOZ and immobilized on a gold chip.

FIG. 23B is a diagram showing derivation of the dissociation constant (K_{D}) by measuring binding affinity between the V_{H}-RBD2 and V_{L}-RBD1 combination antibody expressed via pTRIOZ and the PEDV RBD antigen using SPR analysis.

FIG. 24A is a diagram showing microscopic images comparing inhibition of cell infection when Anti-RBD1 IgG was treated at various concentrations to evaluate neutralizing effects (PEDV: 200 TCID₅₀/0.1 ml).

FIG. 24B is a diagram showing quantitative analysis of neutralization efficacy by measuring relative infection rates according to Anti-RBD1 IgG concentration.

FIG. 24C is a diagram showing quantitative comparison of minimum neutralizing concentrations to evaluate neutralizing effects of Anti-RBD1 IgG.

### DETAILED DESCRIPTIONS

Hereinafter, embodiments of the present invention will be described in detail with reference to the accompanying drawings. The present invention may be modified in various ways and may have various forms, and specific embodiments are illustrated in the drawings and described in detail herein. However, this is not intended to limit the present invention to the specific disclosed embodiments, and it should be understood that the present invention includes all modifications, equivalents, and substitutes falling within the spirit and technical scope of the invention. In describing the drawings, like reference numerals have been used for like elements. In the accompanying drawings, the dimensions of structures are exaggerated for clarity of the present invention.

The terminology used herein is for the purpose of describing particular embodiments only and is not intended to limit the present invention. Singular expressions include plural expressions unless the context clearly indicates otherwise. As used herein, the terms “comprise” or “have” are intended to specify the presence of stated features, numbers, steps, operations, elements, components, or combinations thereof, but do not preclude the presence or addition of one or more other features, numbers, steps, operations, elements, components, or combinations thereof. In the context of this specification, the term “about” may refer to approximately ±1%, ±2%, ±3%, ±4%, ±5%, ±6%, ±7%, ±8%, ±9%, or ±10% of the stated value.

In addition, descriptions relating to one aspect of the present invention may be applied in the same or similar manner to descriptions relating to other aspects.

Unless otherwise defined, all terms used herein, including technical and scientific terms, have the same meanings as commonly understood by a person having ordinary skill in the art to which the present invention pertains. Terms defined in commonly used dictionaries should be interpreted as having meanings consistent with their meanings in the context of the relevant art and should not be interpreted in an idealized or overly formal sense unless expressly defined herein.

The peptide according to an embodiment of the present invention can induce production of antibodies against porcine epidemic diarrhea virus (PEDV) in vivo. In one embodiment, the peptide may comprise one or more peptide sequences selected from the group consisting of the peptide sequence of SEQ ID NO: 1, the peptide sequence of SEQ ID NO: 2, the peptide sequence of SEQ ID NO: 3, and the peptide sequence of SEQ ID NO: 4.

In the context of the present specification, the term “peptide” refers to a short chain composed of a specific amino acid sequence and having the ability to selectively bind to a viral antigen. Such peptides may play a key role in antibody formation and induction of immune responses, and may be used to enhance binding affinity to specific viral regions. Through this, viral infection may be blocked or inhibited, and the peptides may be applied as a strategy to enhance vaccine efficacy. In addition, through various sequence modifications, peptide stability and immune activation may be regulated, thereby expanding responsiveness to viral mutations.

In the context of the present specification, the terms “antibody expression” or “immunization” include inducing a host defense response to an external antigen and the resulting production of antibodies. This process is based on mechanisms that recognize specific antigens and activate immune responses, and may affect both cellular immunity and humoral immunity. Antibody expression may be directly induced in infected host cells, thereby helping the immune system strengthen memory responses to the antigen. Immunization may contribute not only to defense against pathogens but also to maintenance of long-term protective effects after vaccination.

Meanwhile, the nucleic acid according to an embodiment of the present invention may encode the peptide. In the context of the present specification, the term “nucleic acid” refers to a molecule that stores and transmits genetic information and includes DNA (deoxyribonucleic acid) and RNA (ribonucleic acid). Such nucleic acids may provide genetic information necessary for synthesis of proteins or peptides based on nucleotide sequences specifying particular amino acid sequences. They may also be used as tools in recombinant technology or synthetic biology to express specific proteins or implement desired biological functions. Through sequence manipulation of nucleic acids, it may be possible to accurately express antigens capable of inducing immune responses.

In the context of the present specification, “encoding” refers to the process of transmitting genetic information to generate a peptide composed of a specific amino acid sequence. This includes enabling nucleic acid sequences to be expressed as peptides through transcription and translation. In this process, nucleotide sequences of genes are transcribed into mRNA and subsequently translated into amino acid sequences at ribosomes. Through this process, accurate synthesis of peptides capable of binding specific antigens becomes possible and plays a key role in antigen expression and induction of immune responses in vaccine development.

As known in the art, combinations of nucleic acids encoding amino acids included in a peptide may vary widely. Accordingly, the present invention encompasses not only the peptides described above but also nucleic acids encoding the peptides, including theoretically 3,623,878,656 nucleic acid sequences encoding SEQ ID NO: 1, 130,459,631,616 nucleic acid sequences encoding SEQ ID NO: 2, 37,572,373,905,408 nucleic acid sequences encoding SEQ ID NO: 3, and 16,307,453,952 nucleic acid sequences encoding SEQ ID NO: 4. Such diversity arises from codon variability. A codon is a sequence of three nucleotides encoding one amino acid, and multiple codons may encode the same amino acid, allowing various nucleic acid sequences to encode the same peptide sequence. This increases flexibility of the present invention and facilitates selection of nucleic acid sequences optimized for specific biological systems or applications. For example, nucleic acid sequences may be optimized by considering codon usage that is more efficiently translated in a particular organism, thereby improving expression levels and stability of the peptide.

Meanwhile, the recombinant expression vector according to an embodiment of the present invention may comprise the nucleic acid. In the context of the present specification, a recombinant expression vector refers to a DNA molecule engineered for gene expression. It delivers a gene into a new host cell and enables translation of the gene into a protein. The recombinant expression vector may include a specific gene or DNA sequence to induce production of a specific protein in a host cell. The vector may include a promoter, an origin of replication, a selection marker, and other regulatory sequences for expression of the inserted gene. These elements enhance efficiency and stability of gene expression and contribute to optimization for specific cell types or conditions. Recombinant expression vectors play a key role in biological research and pharmaceutical development, particularly in vaccine development, enabling effective development of prevention and treatment strategies for various diseases.

Meanwhile, the cell according to an embodiment of the present invention may be transformed with the recombinant expression vector. In the context of the present specification, “transformation” refers to introduction of externally derived genetic material into a cell. Through this process, cells acquire new genetic characteristics, resulting in changes in physiological, morphological, or genetic properties. Transformation is widely used in gene expression, protein production, and biotechnological research and applications. Such transformed cells induce production of specific proteins or peptides encoded by recombinant expression vectors and may be used for medical or research purposes. In particular, these cells play an important role in vaccine development and contribute to development of new therapeutics and diagnostic methods.

In one embodiment, the cell may comprise one or more cells selected from the group consisting of an animal cell, a plant cell, yeast, Escherichia coli, and an insect cell. In one embodiment, the cell may comprise one or more cells selected from the group consisting of COS7 (monkey kidney) cells, NSO cells, SP2/0 cells, Chinese hamster ovary (CHO) cells, WI38 cells, baby hamster kidney (BHK) cells, MDCK cells, myeloma cell lines, HuT78 cells, HEK293 cells, Escherichia coli, Bacillus subtilis, Streptomyces sp., Pseudomonas sp., Proteus mirabilis, Staphylococcus sp., Aspergillus sp., Pichia pastoris, Saccharomyces cerevisiae, Schizosaccharomyces sp., and Neurospora crassa.

Selection of the cell may be based on cell types suitable for expression and production of specific proteins or peptides. Among various cell types, COS7, NSO, SP2/0, CHO, WI38, BHK, MDCK, myeloma cell lines, HuT78, and HEK293 cells are of human or animal origin and are widely used for protein expression and production. In addition, bacterial types such as Escherichia coli, Bacillus subtilis, Streptomyces sp., Pseudomonas sp., Proteus mirabilis, and Staphylococcus sp. may be used for protein production. Furthermore, fungi and yeast such as Aspergillus sp., Pichia pastoris, Saccharomyces cerevisiae, Schizosaccharomyces sp., and Neurospora crassa may also be suitable for protein production. These cells may be used to optimize purification, modification, or functional activity of proteins based on their characteristics, and may be selected according to production and functional properties of specific proteins or peptides for efficient protein production in biomedical and diagnostic fields.

Meanwhile, a vaccine material for porcine epidemic diarrhea virus according to an embodiment of the present invention may comprise a ferritin protein and a peptide bound to an outer surface of the ferritin protein. In one embodiment, the peptide may be the peptide according to the embodiments described above.

In the context of the present specification, the term “ferritin protein” refers to a spherical nanostructure that stores and stabilizes metal ions and primarily functions to safely store iron ions. Due to this structural property, ferritin may be used as a platform capable of encapsulating various materials within its interior or binding specific molecules on its outer surface. In particular, ferritin has structural stability and repetitive subunit arrangement suitable for antigen binding and has attracted attention in immunological applications. Accordingly, ferritin may be used to effectively arrange and present antigens in vaccine development, thereby contributing to increased strength and durability of immune responses.

In one embodiment, the ferritin protein may comprise an H-ferritin protein. In the context of the present specification, the term “H-ferritin protein” refers to a protein primarily involved in iron storage and capable of maintaining intracellular iron homeostasis. This protein forms a nanoscale spherical structure and may be used as a platform for binding or delivering various biomolecules. In particular, it may provide a surface structure capable of efficiently presenting antigens and creating a favorable environment for inducing immune responses. These characteristics may contribute to stable delivery of vaccine antigens and sustained immune stimulation.

Meanwhile, a vaccine composition for porcine epidemic diarrhea virus according to an embodiment of the present invention may comprise the vaccine material for porcine epidemic diarrhea virus. The vaccine composition includes a specific peptide bound to an H-ferritin protein, thereby forming a structure capable of efficiently presenting antigens to the immune system. This structural feature may contribute to increasing the strength and durability of immune responses and may help activate various antibody generation pathways. In addition, it may promote immune responses against mutated viral strains, thereby providing broader protective effects. Through this, the stability of infection prevention may be improved and economic losses caused by PEDV may be reduced.

The vaccine composition according to an embodiment of the present invention does not exclude addition of other materials in addition to the vaccine material for porcine epidemic diarrhea virus. Non-limiting examples of materials that may be included in the composition include aluminum compounds acting as adjuvants, oil-based adjuvants such as Quil A or Montanide that enhance immune responses, stabilizers (e.g., sugars and trehalose) to improve stability, surfactants (e.g., polysorbate 80) for protein stabilization, preservatives (e.g., thimerosal) to extend storage life, buffers (e.g., phosphate buffers) for pH control, solvents (e.g., glycerol) for improving solubility, absorption enhancers (e.g., chitosan) that may promote in vivo absorption, and antibiotics (e.g., gentamicin) that may provide antimicrobial effects. These components may contribute to improving vaccine stability, immunogenicity, storage properties, and administration efficiency.

Meanwhile, a method for immunizing against porcine epidemic diarrhea virus according to an embodiment of the present invention comprises administering the vaccine composition for porcine epidemic diarrhea virus to an animal. By administering the composition to an animal, the immune system may recognize the antigen contained in the vaccine composition and induce an immune response. This may promote antibody production and activate both cellular and humoral immune responses. In particular, induction of antibody formation by the administered antigen may promote immune memory against PEDV and provide protective effects upon subsequent exposure to the virus. In addition, increased strength and durability of immune responses may provide long-term protective effects. In this process, adjuvants or absorption enhancers included in the composition may further enhance vaccine efficacy.

Hereinafter, examples of the present invention will be described. However, the following examples are merely illustrative of certain embodiments of the present invention, and the scope of the present invention is not limited to the following examples.

### [Fv Antibody Library Platform-Based Antigen Discovery for a PEDV Vaccine (Platform-1)]

### [Cloning and Expression of a Target Antigen for Fv Antibody Library Screening]

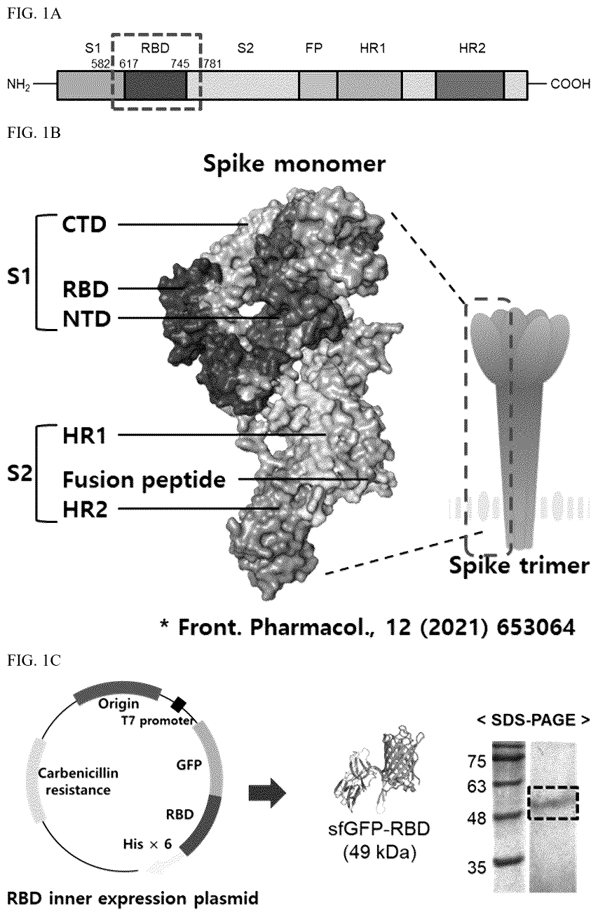

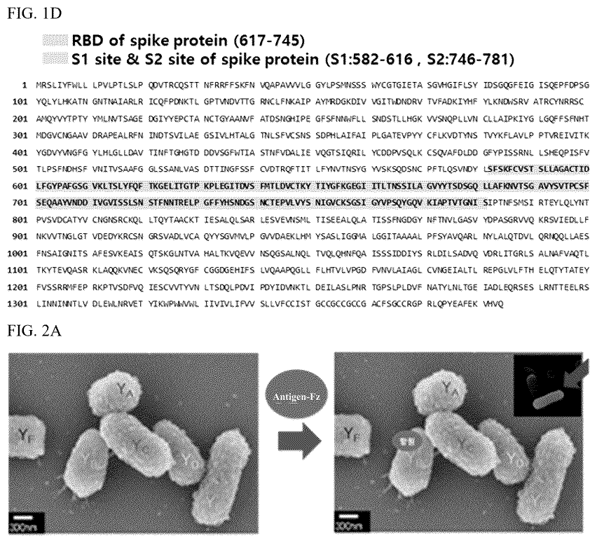

In the present invention, the receptor-binding domain (RBD) of the porcine epidemic diarrhea virus (PEDV) spike protein, which corresponds to the host cell-binding region, is expressed as a probe for screening an Fv antibody library. In this process, a portion of the PEDV spike protein corresponding to amino acids 617 to 745, consisting of 200 amino acids in total, is selected and expressed. The screening probe is expressed in a form fused with the fluorescent protein sfGFP, thereby facilitating the screening process through fluorescence signals. This fusion protein has a molecular weight of approximately 49 kDa and can be used to improve screening efficiency (FIGS. 1A-1D).

### [Fv Antibody Library Screening]

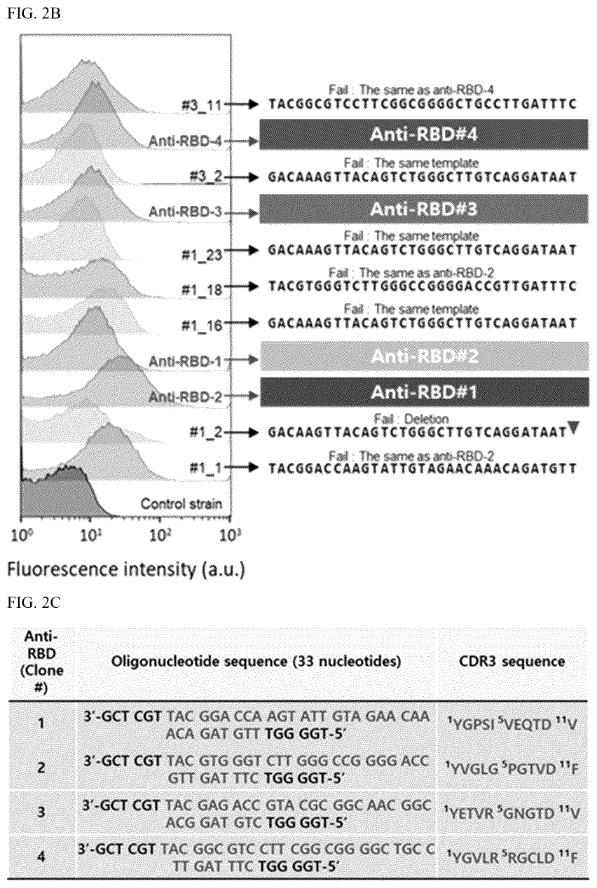

In the present invention, an Fv antibody library capable of binding to the RBD is screened using a PEDV spike protein fused with a fluorescent protein (fluorescent-RBD). During the screening process, candidate *Escherichia coli* clones bound to fluorescent-RBD are separated using fluorescence-activated cell sorting (FACS), and four final *E. coli* clones having normal gene sequences are selected through gene sequence analysis. From these final clones, gene sequences and corresponding amino acid sequences capable of binding to fluorescent-RBD are obtained, which can contribute to analysis of antibody characteristics and evaluation of applicability as vaccine antigens (FIGS. 2A-2C).

### [Analysis of Fv Antibody Binding Affinity]

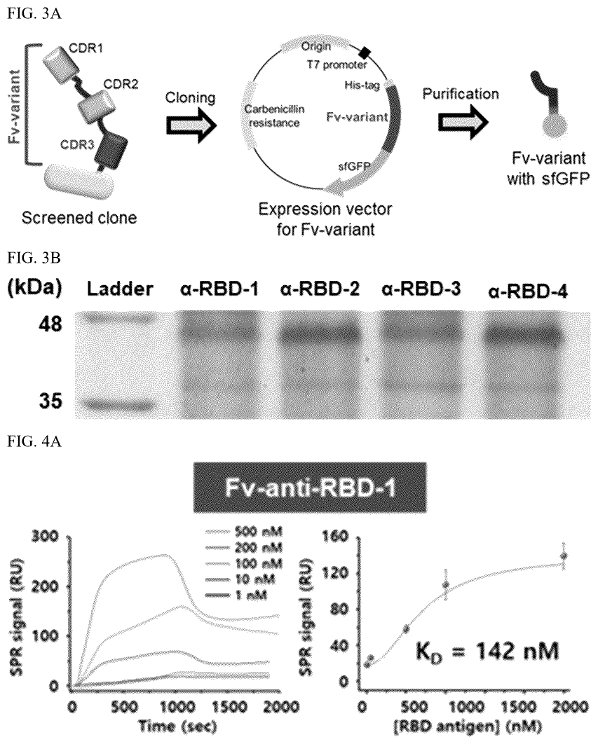

In the present invention, amino acid sequences binding to fluorescent-RBD obtained from the four final clones through Fv antibody library screening are individually expressed as Fv antibodies. These Fv antibodies correspond to the heavy chain hypervariable region (VHH) of IgG and are expressed to include three complementarity determining regions (CDRs) and four framework regions (FRs). Expression of the Fv antibodies is confirmed by SDS-PAGE, and the molecular weight is measured to be approximately 40.6 kDa (FIGS. 3A and 3B).

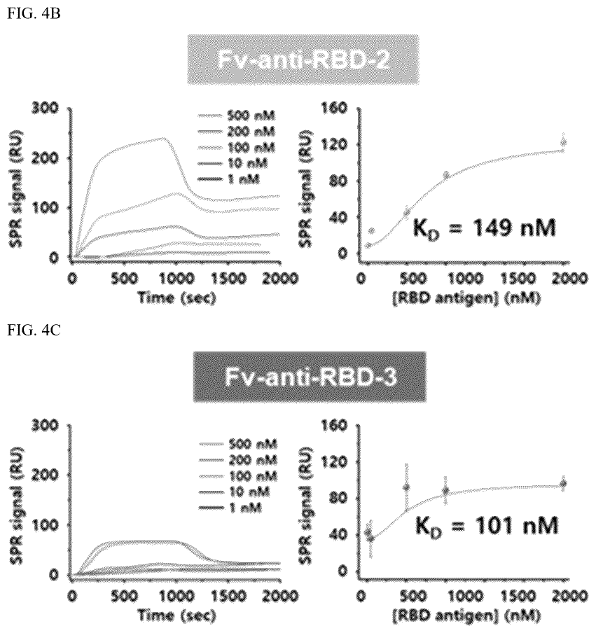

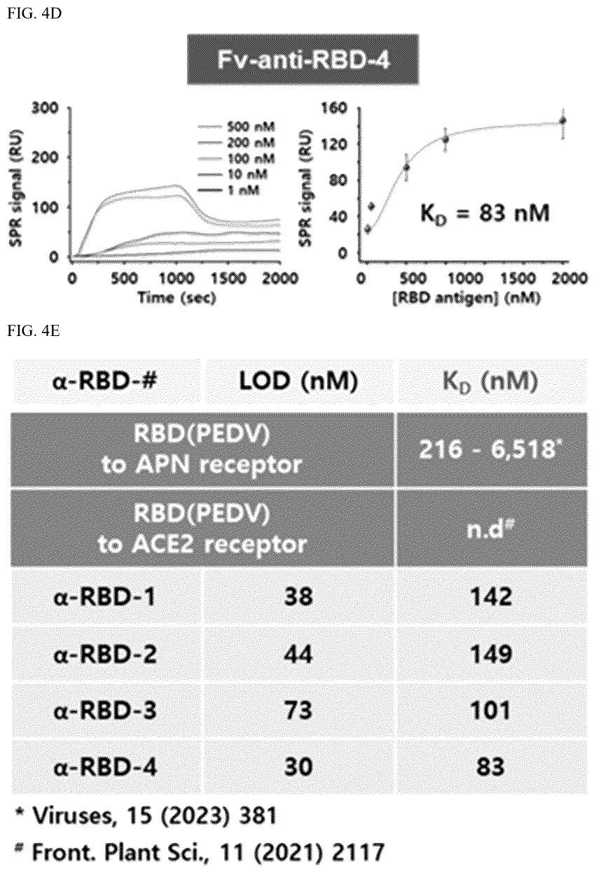

The binding affinity (K_{D}) of the expressed Fv antibodies to the PEDV spike protein (fluorescent-RBD) is measured using an SPR biosensor. For this purpose, Fv antibodies are immobilized on the SPR biosensor surface, fluorescent-RBD is treated at various concentrations, and binding affinity is calculated through signal analysis (FIGS. 4A-4E). The results show that all four Fv antibodies exhibit binding affinities in the range of approximately 100-150 nM. These values are comparable to the known binding affinity of PEDV to the host cell Aminopeptidase-N(APN) receptor (≥100 nM), suggesting that the Fv antibodies obtained in the present invention have strong binding affinity to the PEDV spike protein.

### [Antigen Discovery for a PEDV Vaccine]

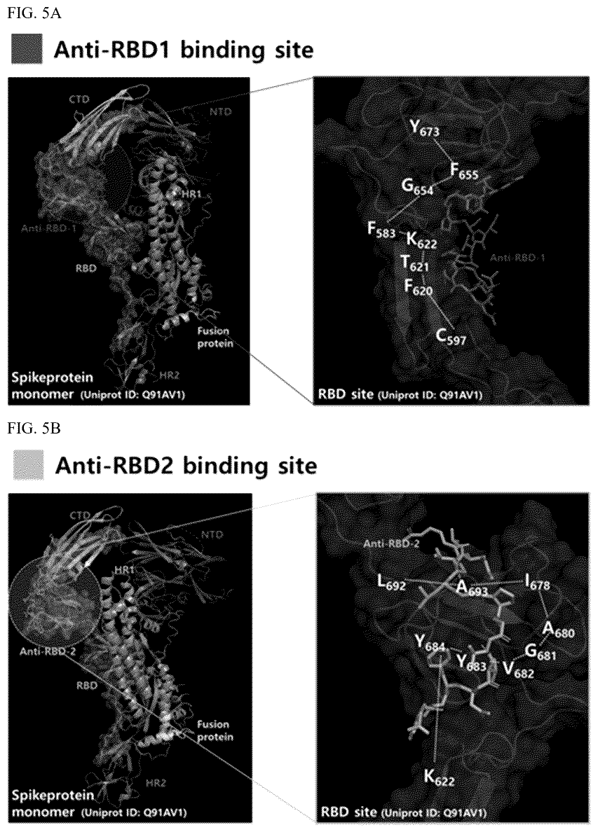

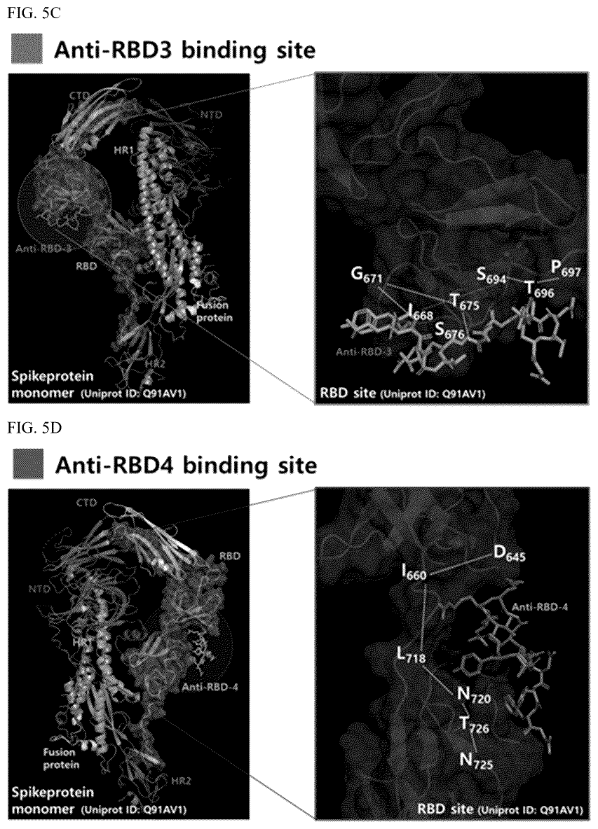

In the present invention, docking simulations are performed to discover vaccine antigens using the four expressed Fv antibodies that strongly bind to the PEDV spike protein. Regions of the PEDV spike protein bound by the four screened amino acid sequences are analyzed to identify regions that can be used as vaccine antigens. Docking simulations are performed using AutoDock Vina (version 1.1.2), and PEDV spike protein (PDB No.: Q91AV1) and the four amino acid sequences that strongly bind thereto are used as inputs. The results indicate that the four amino acid sequences bind to different regions of the PEDV spike protein, and each binding site is locally concentrated (FIGS. 5A-5D).

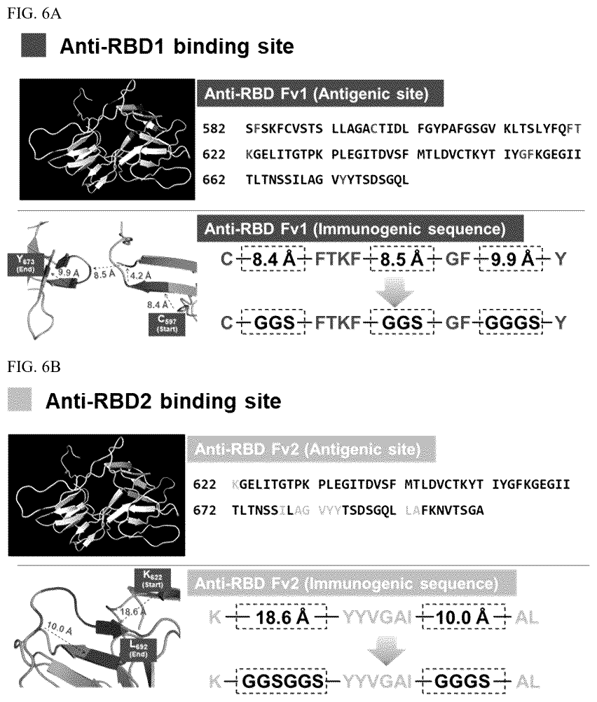

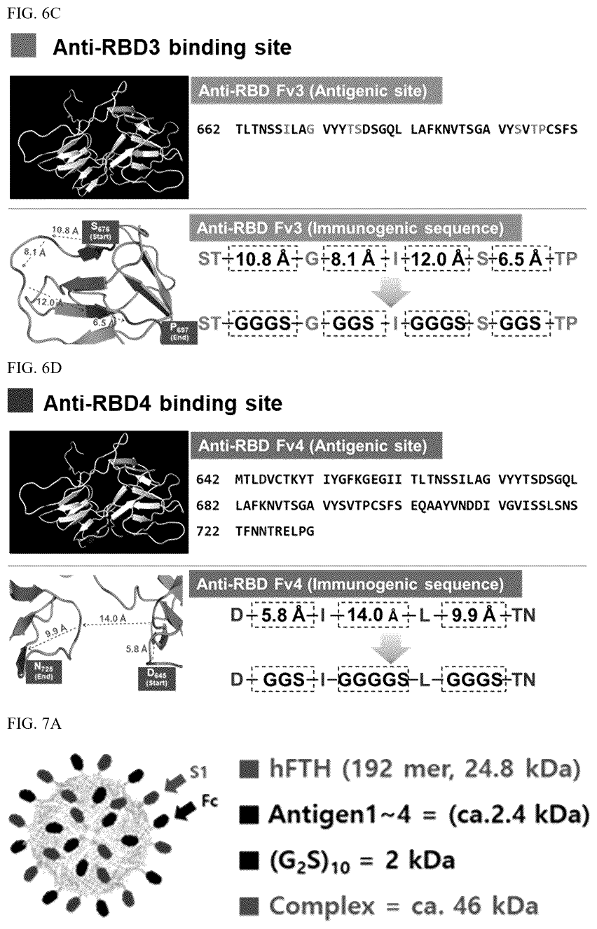

Based on the docking simulation results, the binding regions of the four amino acid sequences are reconstructed into single-domain sequences for expression as vaccine antigens. Distances between Fv antibody binding sites are calculated from the simulation model, and non-immunogenic amino acid sequences (glycine-glycine-serine modules) are inserted to maintain these distances, thereby completing four single-domain vaccine antigen sequences (FIGS. 6A-6D).

### [[PEDV Vaccine Development Using a Ferritin Complex Platform Co-Expressing Antigens (Platform-2)]]

### [Ferritin Complex Expression]

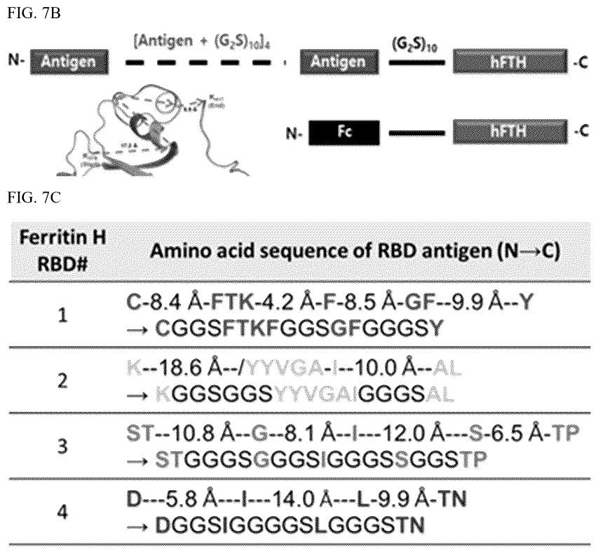

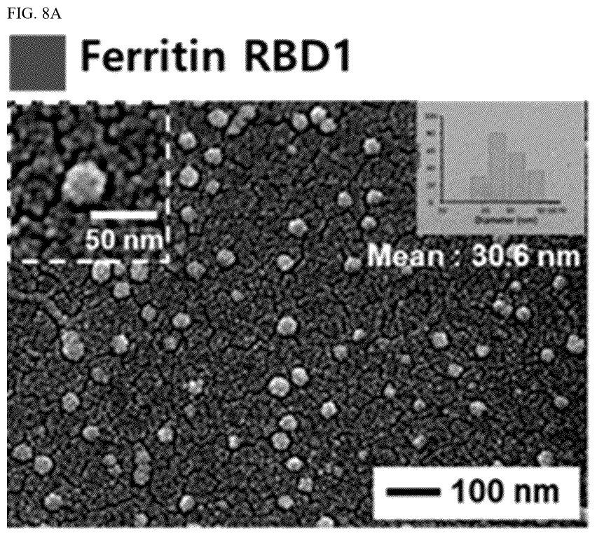

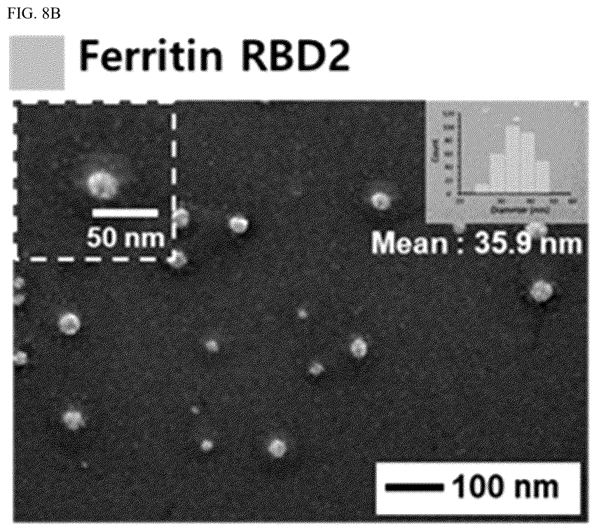

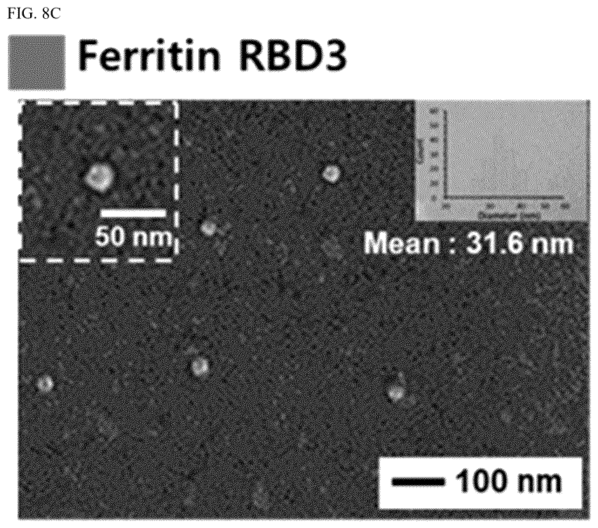

In the present invention, ferritin complexes are expressed by linking single-domain sequences of the four vaccine antigens obtained from docking simulations to ferritin. Each vaccine antigen sequence is linked to ferritin (24.8 kDa) to form a ferritin complex, and the vaccine antigen sequences are connected in a tandem repeat form repeated five times to construct the complex. The molecular weight of the ferritin complex generated in this manner is approximately 46 kDa. Through this method, four ferritin-vaccine antigen complexes are produced, each comprising a different antigen sequence, and the complexes provide a structural basis capable of inducing immune responses as vaccine antigens (FIGS. 7A-7C).

### [Preparation of Ferritin Complex-Based Vaccine Particles]

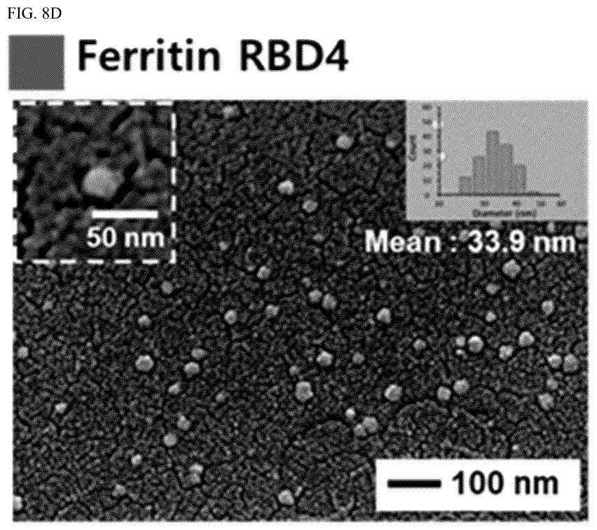

In the present invention, four ferritin-vaccine antigen complexes in which vaccine antigen sequences are bound are expressed and purified, followed by preparation of ferritin-vaccine antigen particles through a self-assembly process. The particles formed in this process were confirmed to have a spherical shape by scanning electron microscopy (SEM), and dynamic light scattering (DLS) analysis showed a uniform diameter of approximately 30 nm (FIGS. 8A-8D).

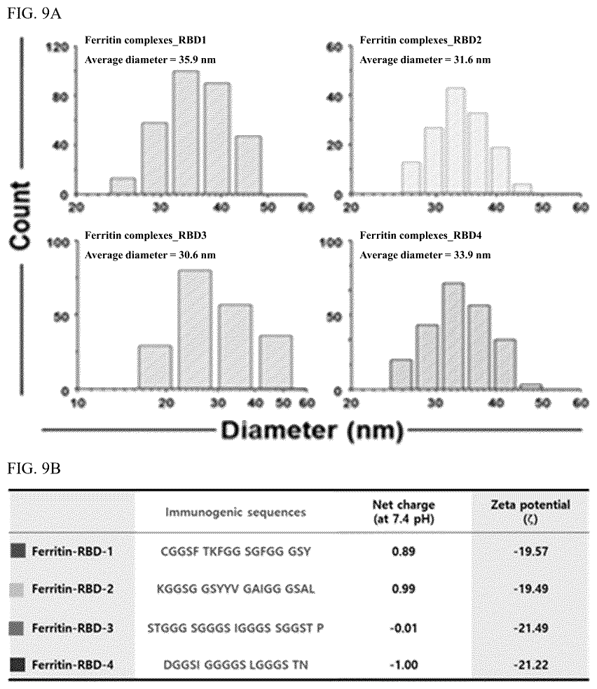

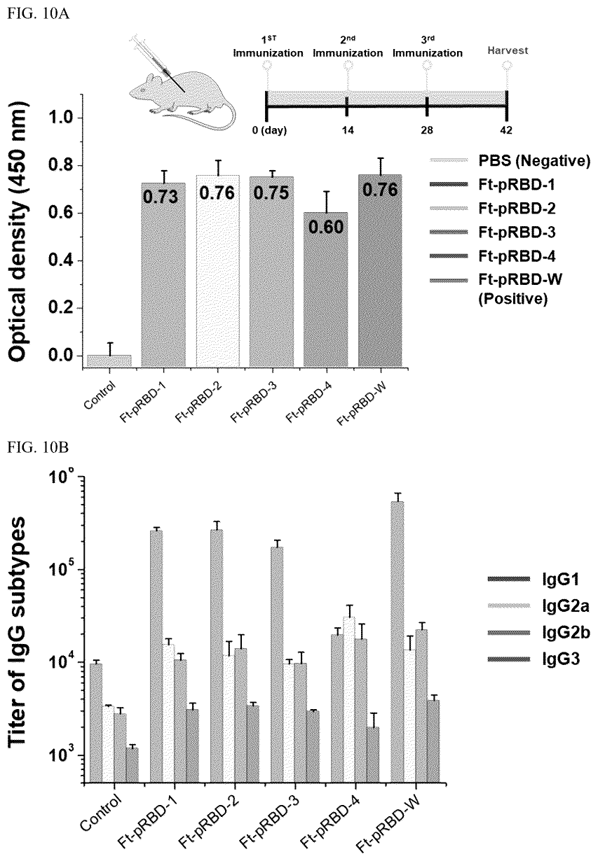

In addition, the four ferritin—vaccine antigen complexes exhibited consistent particle diameters, and their surface properties were analyzed. As a result, each complex maintained a net charge between −1 and 1, and the zeta potential was measured to be between −21.5 and −19.5. These results indicate that the stability and physicochemical properties of the complexes may function effectively in an immunostimulatory environment (FIGS. 9A and 9B).

### [[Neutralization Evaluation of PEDV Vaccine]]

### [Analysis of Immune Responses Induced by Ferritin Complex-Based Vaccine]

Vaccination and serum collection

In the present invention, immune responses were induced by administering four ferritin-vaccine antigen complex vaccines to mice. Immunization was performed in three female mice aged five weeks. Booster shots were administered at weeks 2 and 4 after the initial immunization. At each immunization, 100 μg of the ferritin complex vaccine was injected to enhance the immune response. After repeated immunizations, antisera were collected at week 6 from the initial immunization to evaluate induction of antibody responses.
Antibody titers in antisera

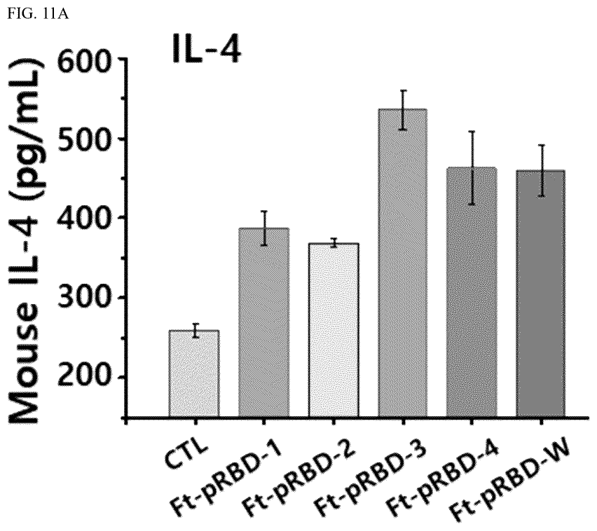

Antibody titers in antisera were measured by immobilizing antigen on a plate, treating with antisera diluted 300-fold, applying a secondary antibody, and measuring the response. The results showed that ferritin complex vaccines using the four Fv antibody-binding antigens induced higher antibody production compared with full-length RBD or ferritin complexes containing full-length RBD (FIGS. 10A and 10B).
IgG subtyping in antisera

IgG subtypes in antisera were analyzed by measuring levels of IgG2a, IgG2b, and IgG3 associated with Th1 immune pathways and IgG1 associated with Th2 pathways. The ferritin complex vaccines containing the four Fv antibody-binding antigens showed higher levels of all IgG subtypes compared with full-length RBD or ferritin complexes containing full-length RBD, indicating activation of diverse immune pathways and induction of strong and sustained immune responses.
Cytokine analysis in antisera

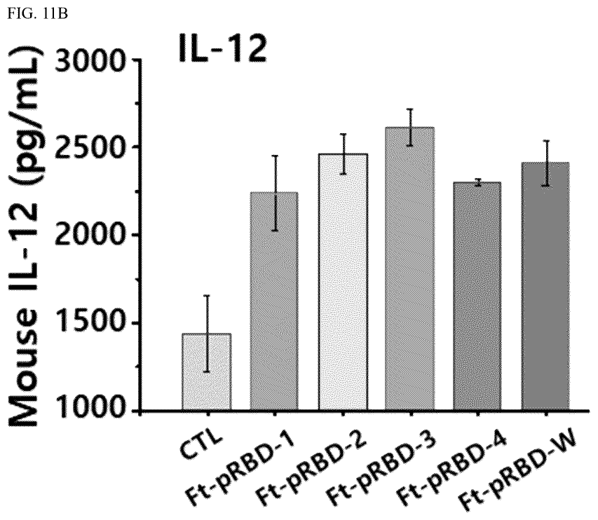

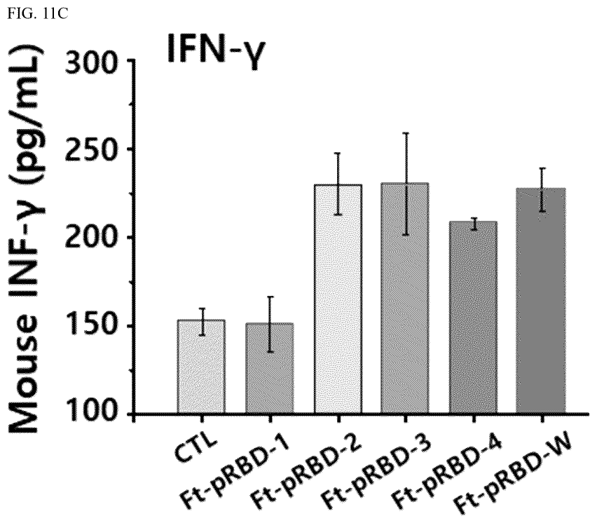

Four cytokines related to Th1 and Th2 immune responses (Th1: IFN-γ, IL-12; Th2: IL-4, TNF-α) were analyzed. All four ferritin complex vaccines activated both Th1 and Th2 immune responses, suggesting the potential to induce strong immune responses (FIGS. 11A-11D). These results indicate that the ferritin complex vaccine can induce effective immune responses and contribute to enhanced protection against PEDV infection.
CD4+/CD8+ T-cell analysis

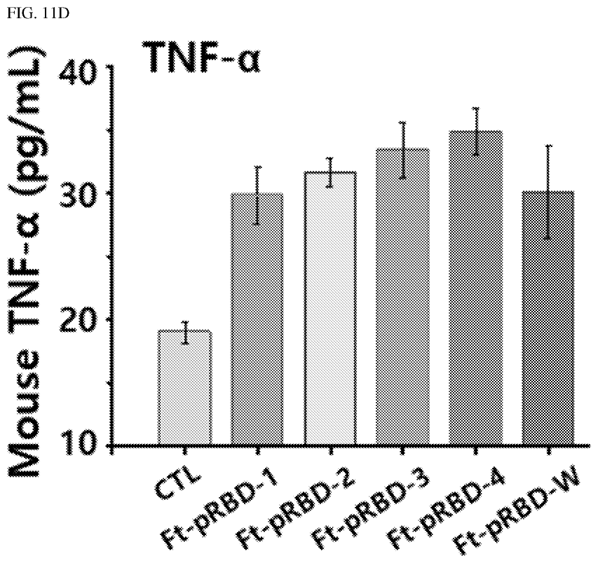

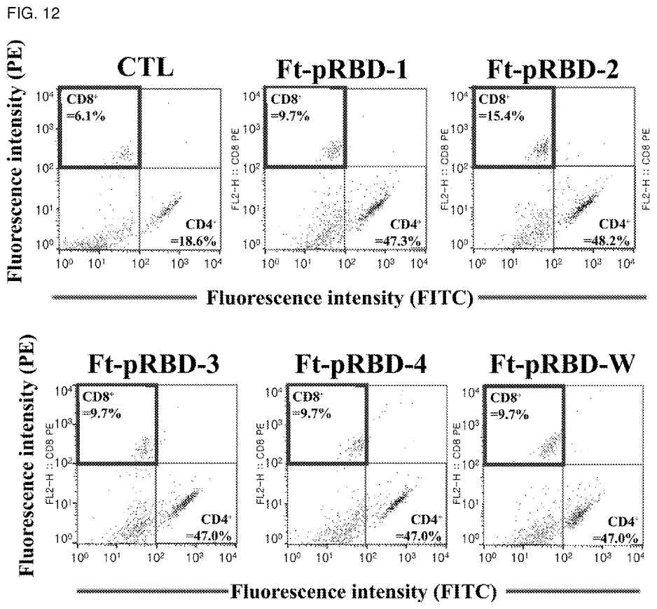

CD4+/CD8+ T-cell analysis was performed using flow cytometry to evaluate T-cell distribution after vaccination. The vaccinated groups showed a marked increase in CD4+ and CD8+ T-cell populations compared with controls, indicating active induction of immune responses (FIG. 12). These results demonstrate that the ferritin complex vaccine can effectively stimulate the immune system and support activation of cellular immune responses for infection prevention and viral clearance.

### [Neutralization Evaluation of Ferritin Complex-Based Vaccine]

Development of PEDV infection assay

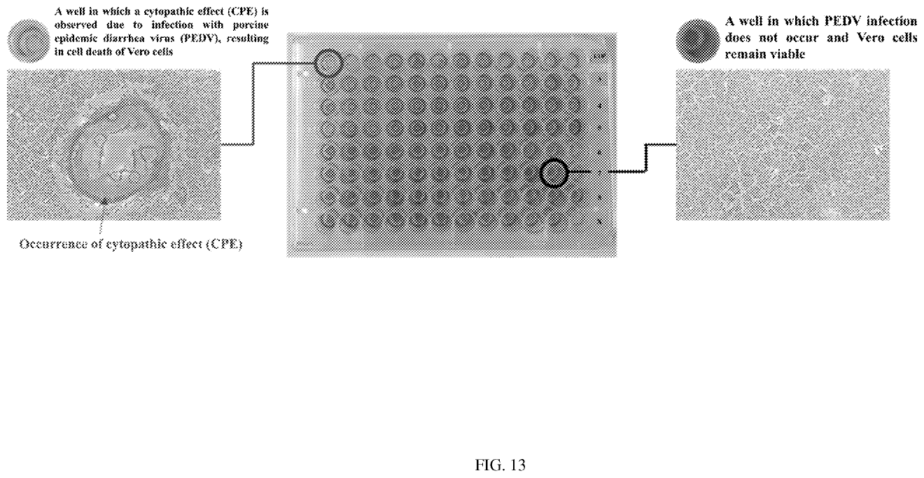

A PEDV infection assay was developed using Vero cells as host cells to evaluate neutralization capacity of antisera. When PEDV infects host cells, cytopathic effects (CPE) and plaque formation occur, enabling quantitative measurement of infection (FIG. 13). PEDV stock was prepared by culturing Vero cells to confluence, infecting with diluted PEDV (KPEDV-9), and culturing for five days to obtain activated PEDV stock for use in infection assays.
Measurement of PEDV infectivity (TCID₅₀/ml)

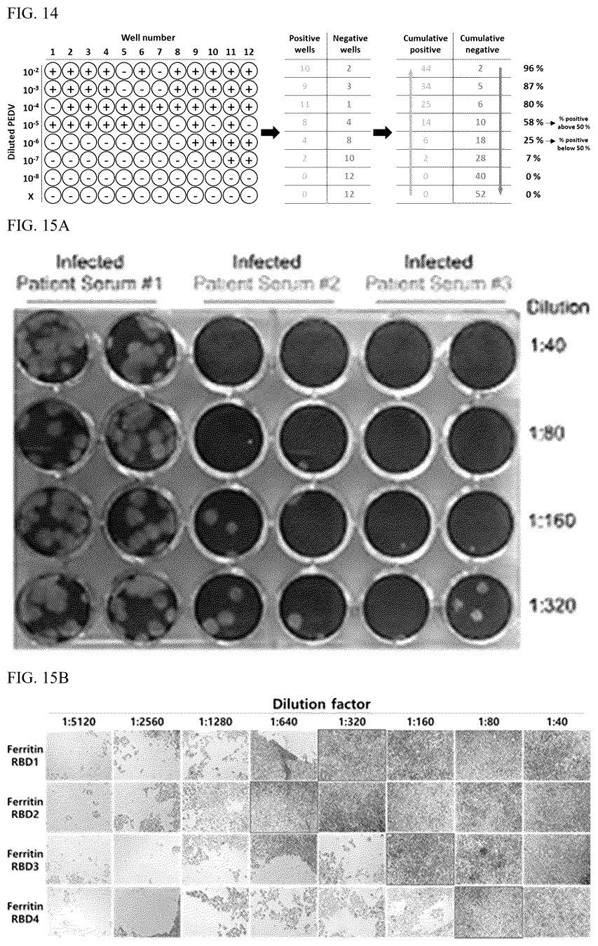

PEDV infectivity was measured by serially diluting PEDV stock in 96-well plates containing Vero cells and culturing for seven days. Cytopathic effects were observed and the Reed-Muench method was applied to calculate infectivity as TCID₅₀/ml. The pseudo-virus stock used for neutralization testing showed an infectivity of 1.7×10⁶ TCID₅₀/ml (FIG. 14).
Neutralization evaluation of ferritin complex vaccine

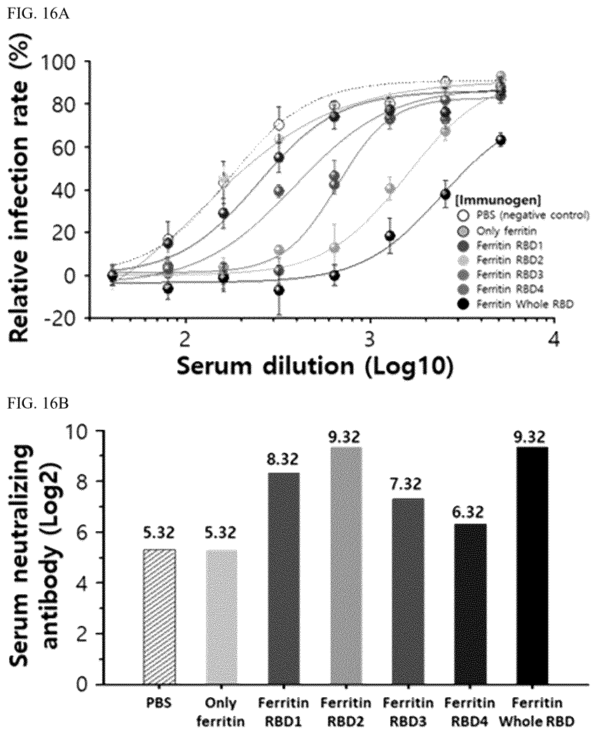

Neutralization assays were performed by serially diluting antisera tenfold and evaluating neutralization at a fixed virus concentration of 200 TCID₅₀/0.1 ml according to regulatory guidelines. Neutralizing antibody titers were calculated as the reciprocal of the highest serum dilution showing no cytopathic effect. The neutralization titers of ferritin complex PEDV vaccines were determined to be 8.32 (Ft-pRBD-1), 9.32 (Ft-pRBD-2), 7.32 (Ft-pRBD-3), and 6.32 (Ft-pRBD-4). The PBS negative control showed a neutralization titer of 6.32, while the positive control containing full-length RBD bound to ferritin showed the highest titer of 9.32. These results indicate that ferritin complex vaccines induce neutralizing antibodies, and the ferritin complex containing Fv-2 exhibited neutralization comparable to the full-length RBD ferritin complex (FIGS. 16A and 16B).

### [[Development of Neutralizing Antibodies Against PEDV]]

### [Fv Antibody Neutralization Evaluation]

Analysis of Fv antibody binding affinity

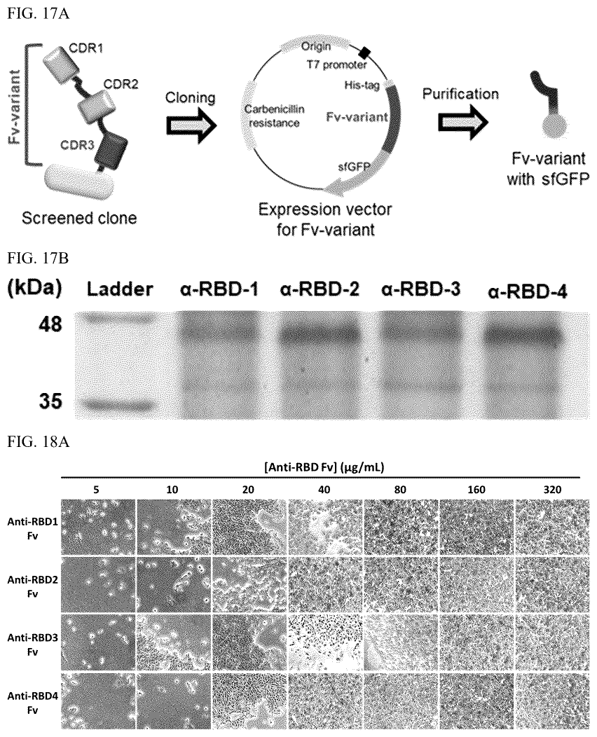

Binding affinity analysis of Fv antibodies was performed by expressing amino acid sequences binding to fluorescent-RBD from the four final clones obtained through Fv antibody library screening as individual Fv antibodies. These Fv antibodies correspond to the heavy chain hypervariable region (VHH) of IgG and were expressed to include three complementarity determining regions (CDRs) and four framework regions (FRs). Expression of the antibodies was confirmed by SDS-PAGE, and the molecular weight was measured to be approximately 40.6 kDa (FIGS. 17A and 17B). The binding affinity analysis showed that the four expressed Fv antibodies exhibited binding affinities in the range of approximately 100-150 nM. These values are comparable to the known binding affinity between PEDV and the host cell Aminopeptidase-N(APN) receptor (≥100 nM), suggesting that the Fv antibodies obtained through screening have strong binding capability to the PEDV spike protein.
Neutralization evaluation of Fv antibodies

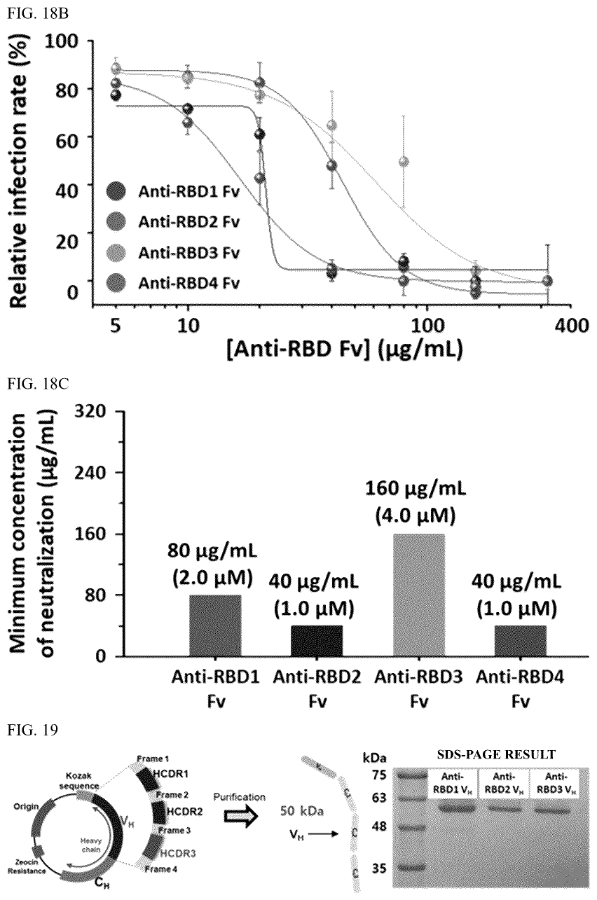

Neutralization testing of Fv antibodies was performed by serially diluting the four Fv antibodies tenfold at a fixed virus concentration of 200 TCID₅₀/0.1 ml and calculating the minimum neutralizing concentration. The analysis was conducted according to regulatory guidelines. The minimum neutralizing concentrations were determined to be 80 μg/ml (Fv-anti-RBD-1), 40 μg/ml (Fv-anti-RBD-2), 160 μg/ml (Fv-anti-RBD-3), and 40 μg/ml (Fv-anti-RBD-4). These results suggest that the Fv antibodies may function as neutralizing antibodies capable of inhibiting PEDV infection. In particular, Fv antibody 2 and Fv antibody 4 showed effective neutralization at lower concentrations (FIGS. 18A-18C).

### [VH Antibody Neutralization Evaluation]

Analysis of VH antibody binding affinity

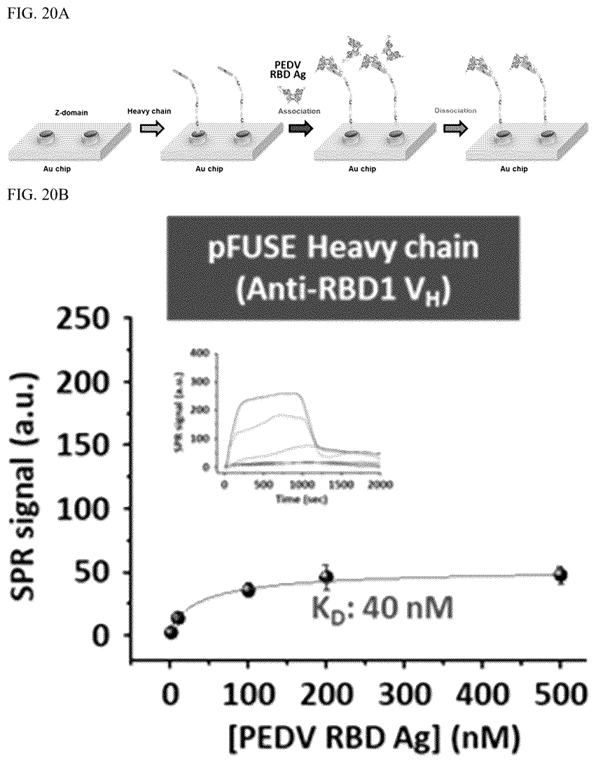

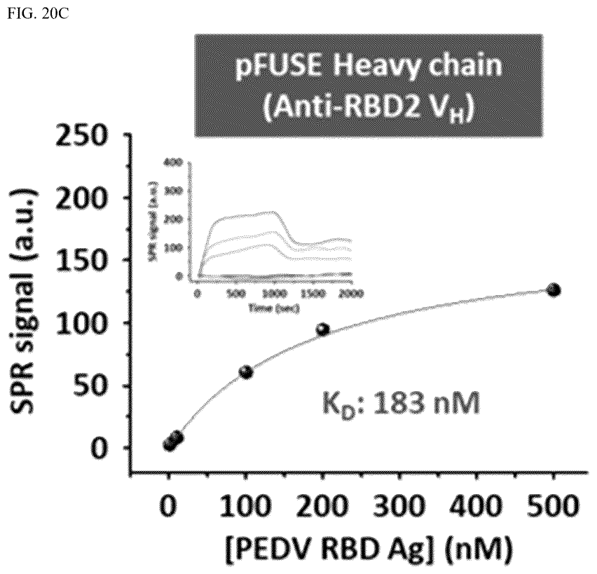

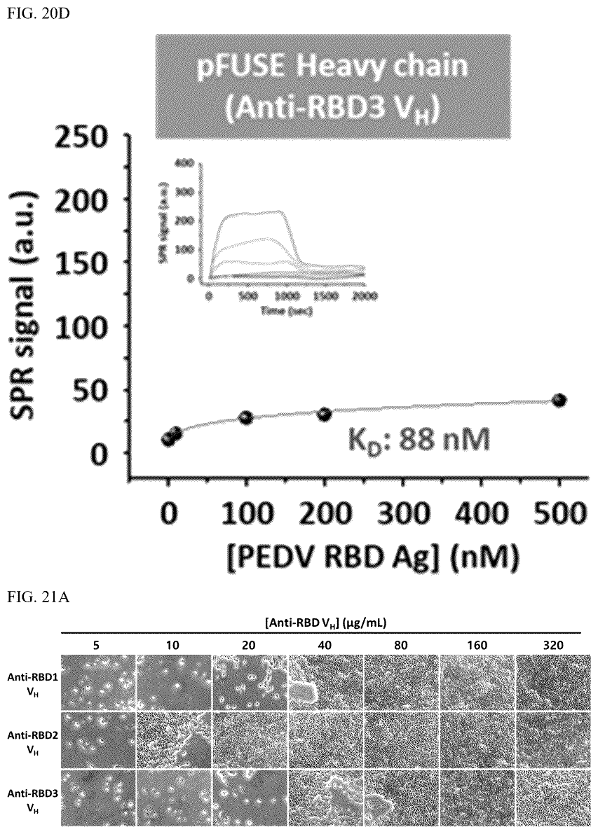

VH antibody binding affinity analysis was performed based on amino acid sequences binding to fluorescent-RBD obtained from the four final clones. Each sequence was expressed as an individual VH antibody by insertion into a pFUSE vector containing the IgG heavy chain hypervariable region (VHH). Expression was confirmed by SDS-PAGE, and the molecular weight was measured to be approximately 50 kDa (FIG. 19). Binding affinity to the PEDV spike protein was measured using an SPR biosensor (FIGS. 20A-20D). The measured affinities ranged from 40 to 180 nM, indicating strong binding comparable to the known PEDV-APN receptor interaction.
Neutralization evaluation of VH antibodies

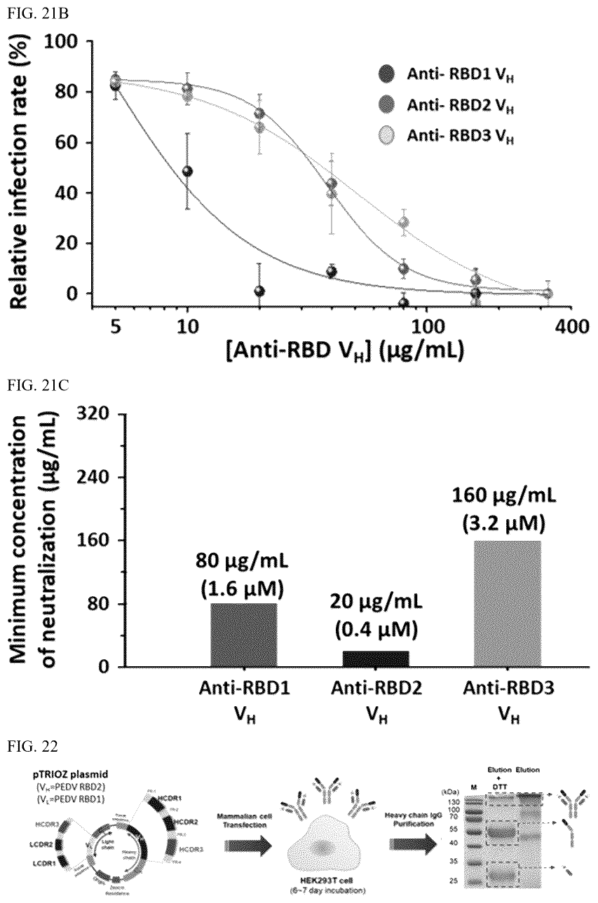

Neutralization testing of VH antibodies was performed at a fixed virus concentration of 200 TCID₅₀/0.1 ml using three VH antibodies diluted tenfold. The minimum neutralizing concentrations were determined to be 80 μg/ml (VH antibody 1), 20 μg/ml (VH antibody 2), and 160 μg/ml (VH antibody 3). VH antibody 2 exhibited the strongest neutralization at the lowest concentration (FIGS. 21A-21C).

### [IgG Antibody Neutralization Evaluation]

Analysis of IgG antibody binding affinity

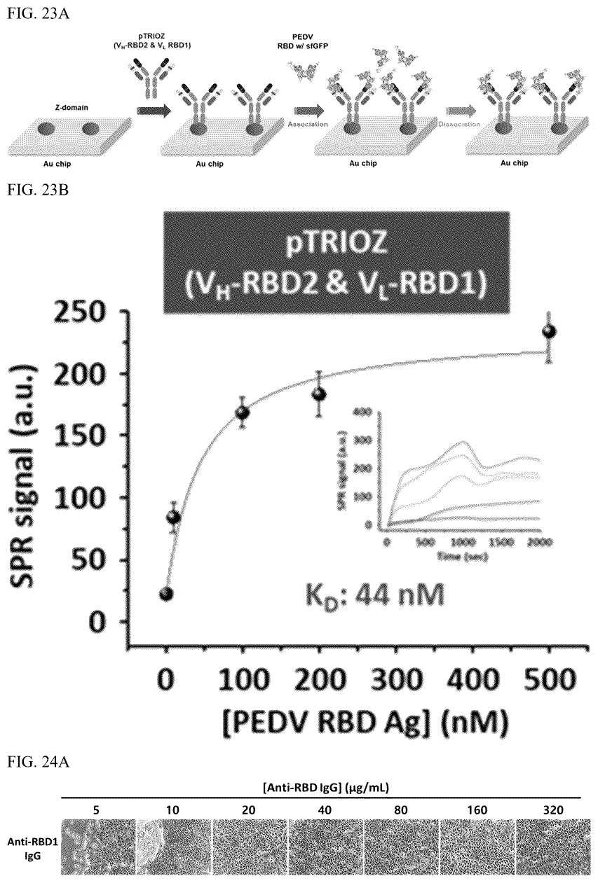

IgG antibody binding affinity analysis was performed based on amino acid sequences binding to fluorescent-RBD obtained from the four final clones. The IgG antibody was designed to include the CDR3 sequence of Fv antibody 2 in the heavy chain and the CDR3 sequence of Fv antibody 1 in the light chain and was expressed using a pTRIOZ vector. Expression was confirmed by SDS-PAGE, and the molecular weight was approximately 150 kDa (FIG. 22). SPR analysis measured the binding affinity to PEDV RBD at approximately 44 nM (FIGS. 23A and 23B), indicating strong binding comparable to the PEDV-APN interaction.
Neutralization evaluation of IgG antibody

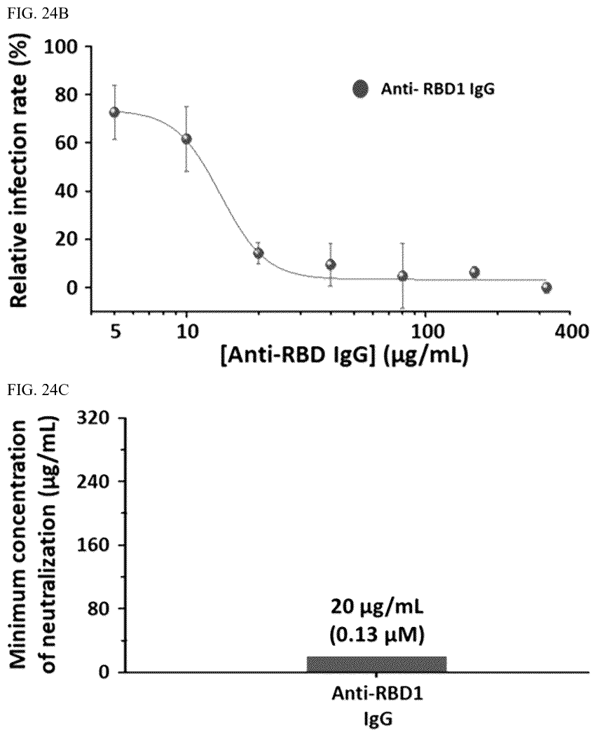

Neutralization testing was performed using the IgG antibody with high binding affinity at a fixed virus concentration of 200 TCID₅₀/0.1 ml. The antibody was serially diluted twofold to determine the minimum neutralizing concentration. The minimum neutralizing concentration was determined to be 20 μg/ml (IgG antibody 1), indicating effective inhibition of PEDV infection (FIGS. 24A-24C).

Although the preferred embodiments of the present invention have been described above, those skilled in the art will understand that various modifications and changes can be made without departing from the spirit and scope of the invention as defined in the appended claims.

What is claimed is:

1. A peptide that induces production of antibodies against porcine epidemic diarrhea virus (PEDV) in vivo, the peptide comprising an amino acid sequence selected from the group consisting of: (a) the amino acid sequence of SEQ ID NO: 1; (b) the amino acid sequence of SEQ ID NO: 2; (c) the amino acid sequence of SEQ ID NO: 3; and (d) the amino acid sequence of SEQ ID NO: 4.

2. A nucleic acid encoding the peptide according to claim 1.

3. A recombinant expression vector comprising the nucleic acid according to claim 2.

4. A cell transformed with the recombinant expression vector according to claim 3.

5. The cell according to claim 4,
wherein the cell comprises one or more cells selected from the group consisting of an animal cell, a plant cell, yeast, Escherichia coli, and an insect cell.

6. The cell according to claim 5,
wherein the cell comprises one or more cells selected from the group consisting of COS7 (monkey kidney) cells, NSO cells, SP2/0 cells, Chinese hamster ovary (CHO) cells, WI38 cells, baby hamster kidney (BHK) cells, MDCK cells, myeloma cell lines, HuT78 cells, HEK293 cells, Escherichia coli, Bacillus subtilis, Streptomyces sp., Pseudomonas sp., Proteus mirabilis, Staphylococcus sp., Aspergillus sp., Pichia pastoris, Saccharomyces cerevisiae, Schizosaccharomyces sp., and Neurospora crassa.

7. A vaccine material for porcine epidemic diarrhea virus, comprising:
ferritin protein; and
a peptide bound to an outer surface of the ferritin protein,
wherein the peptide comprises the peptide according to claim 1.

8. The vaccine material for porcine epidemic diarrhea virus according to claim 7,
wherein the ferritin protein comprises H-ferritin protein.

9. A vaccine composition for porcine epidemic diarrhea virus comprising the vaccine material according to claim 7.

10. A method for immunizing against porcine epidemic diarrhea virus, comprising administering the vaccine composition according to claim 9 to an animal.


SEQUENCE LISTING (10 sequences)

SEQ ID NO: 1 - protein, 18 aa, Synthetic construct
CGGSFTKFGGSGFGGSGY

SEQ ID NO: 2 - protein, 19 aa, Synthetic construct
KGGSGGSYYVGAIGGGSAL

SEQ ID NO: 3 - protein, 21 aa, Synthetic construct
STGGGSGGGSIGGGSSGGSTP

SEQ ID NO: 4 - protein, 17 aa, Synthetic construct
DGGSIGGGGSLGGGSTN

SEQ ID NO: 5 - protein, 5 aa, Synthetic construct
TYGIQ

SEQ ID NO: 6 - protein, 17 aa, Synthetic construct
WIHAGTGGTKYSRKFQG

SEQ ID NO: 7 - protein, 11 aa, Synthetic construct
YGPSIVEQTDV

SEQ ID NO: 8 - protein, 11 aa, Synthetic construct
YVGLGPGTVDF

SEQ ID NO: 9 - protein, 11 aa, Synthetic construct
YETVRGNGTDV

SEQ ID NO: 10 - protein, 11 aa, Synthetic construct
YGVLRRGCLDF